(19) United States
(12) Patent Application Publication
(10) Pub. No.: US 2025/0252218 A1
(43) Pub. Date: Aug. 7, 2025

(54) A SYSTEM AND METHOD FOR ANONYMIZING VIDEOS

(71) Applicant: Kartik MANGUDI VARADARAJAN, Del Mar, CA (US)

(72) Inventors: Kartik MANGUDI VARADARAJAN, Del Mar, CA (US); Gokce YILDIRIM, Weehawken, NJ (US)

(73) Assignee: Kartik MANGUDI VARADARAJAN, Del Mar, CA (US)

(21) Appl. No.: 18/854,985

(22) PCT Filed: Apr. 13, 2023
(86) PCT No.: PCT/IB2023/053770

§ 371 Date: Oct. 8, 2024

Related U.S. Application Data
(60) Provisional application No. 63/331,048, filed on Apr. 14, 2022.

(51) Int. Cl. G06F 21/62; G06T 7/73

(52) U.S. Cl. CPC G06F 21/6254; G06T 7/73; G06T 2207/30204

(57) ABSTRACT

A system and method for anonymizing videos is provided. The system comprises of a server, wherein the server comprises one or more processors. The one or more processors are configured to receive a video captured by an imaging device, wherein the video comprises of a plurality of video frames. The one or more processors are configured to analyse each of the video frames captured by the imaging device and detect reference entity in each of the video frames and anonymize at least a portion of each of the video frames in which the reference entity is absent.

### FIELD OF THE INVENTION

The present invention, in general, relates to data anonymization. More specifically, the present invention relates to a system and method for anonymizing videos and other image data.

### BRIEF STATEMENT OF THE PRIOR ART

It is in practice to record different medical and surgical processes, so that it can be used for the purposes of training medical students and other trainees. Such video recordings allow students to get better insights into surgeries done in real world. These recordings can also be used for various other non-educational purposes.

One of the key challenges in the use and analysis of healthcare data, such as video and other image data collected in the operating room, is the need to protect sensitive information including protected health information. Examples of such sensitive information include patient's face, faces of operating room staff (even if partially covered by masks, surgical face shields etc.), identity marks on patients (like tattoos, birth marks, burn marks, scars etc), information displayed on electronic screens within the operating room (for e.g., x-ray images), and text in documents such as patient chart viewed by surgeon prior to start of the surgical case, among others.

The potential disadvantages of prior art systems that use deep learning algorithms for anonymizing videos comprising such sensitive data, is the need to train the algorithm with labelled data pertaining to specific types of sensitive information, like human faces, medical records, identity marks and many more. This requires a quantum of labelled image/video data containing the various sensitive data for the system to learn from and improve its accuracy over time. Further, to ensure the system is effective, it needs to be trained on a diverse range of videos/images that contain such sensitive data, which can be a time-consuming and resource-intensive process.

In light of the foregoing, there is a need for an improved and efficient system that is easy to train, efficient and accurate.

### SUMMARY OF THE INVENTION

In an embodiment, a system and method for anonymizing videos is provided. The system comprises a server, wherein the server comprises one or more processors. The one or more processors are configured to receive a video captured by an imaging device, wherein the video comprises of a plurality of video frames. The one or more processors are configured to analyse each of the video frames captured by the imaging device and detect reference entity in each of the video frames. The one or more processors are configured to anonymize at least a portion of each of the video frames when the reference entity is absent in the video frames.

### BRIEF DESCRIPTION OF DRAWINGS

Embodiments are illustrated by way of example and not limitation in the figures of the accompanying drawings, in which like references indicate similar elements and in which:

FIG. 1 illustrates a block diagram of a system for anonymizing videos, in accordance with an embodiment.

FIGS. 2A-2C depict reference entities 200 employed in different scenarios.

FIG. 3 is a flowchart 300 depicting a method of an embodiment for anonymizing video, in accordance with an embodiment.

FIG. 4 is a flowchart 400 depicting a method of another embodiment for anonymizing video, in accordance with an embodiment.

FIGS. 5-8 illustrate different representation of video frames tagged with a first value or a second value, in accordance with an embodiment.

FIGS. 9 and 10A-10B illustrate anonymization of video frames in different scenarios in the presence of reference entities 200, in accordance with an embodiment.

FIG. 10C illustrate projections of region of interest (ROI) in different dimensional Coordinate systems, in accordance with an embodiment.

FIG. 11 illustrate another embodiment wherein Region of Interest is zoomed in while rest of portions of video frame is anonymized, in accordance with an embodiment.

FIG. 12 illustrate another embodiment depicting a different method of anonymization of videos.

FIG. 13 is a flowchart 1300 depicting a method of tag filtering for video frames, in accordance with an embodiment.

FIGS. 14A-14D illustrate pictorial representation of the method of tag filtering for video frames, in accordance with an embodiment.

FIG. 15 is a flowchart 1500 depicting a method of another embodiment for anonymizing videos, in accordance with an embodiment.

FIGS. 16A-16B illustrate an embodiment for anonymizing videos wherein a 3D space 1602 is projected onto the video frames for analysing the video frames, in accordance with an embodiment.

FIG. 17 illustrate another embodiment depicting anonymization of videos by creating a virtual space 1702.

### DETAILED DESCRIPTION

The following detailed description includes references to the accompanying drawings, which form a part of the detailed description. The drawings show illustrations in accordance with example embodiments. These example embodiments, which may be herein also referred to as “examples” are described in enough detail to enable those skilled in the art to practice the present subject matter. However, it may be apparent to one with ordinary skill in the art, that the present invention may be practised without these specific details. In other instances, well-known methods, procedures and components have not been described in detail so as not to unnecessarily obscure aspects of the embodiments. The embodiments can be combined, other embodiments can be utilized, or structural, logical, and design changes can be made without departing from the scope of the claims. The following detailed description is, therefore, not to be taken in a limiting sense, and the scope is defined by the appended claims and their equivalents.

In this document, the terms “a” or “an” are used, as is common in patent documents, to include one or more than one. In this document, the term “or” is used to refer to a nonexclusive “or,” such that “A or B” includes “A but not B,” “B but not A,” and “A and B,” unless otherwise indicated.

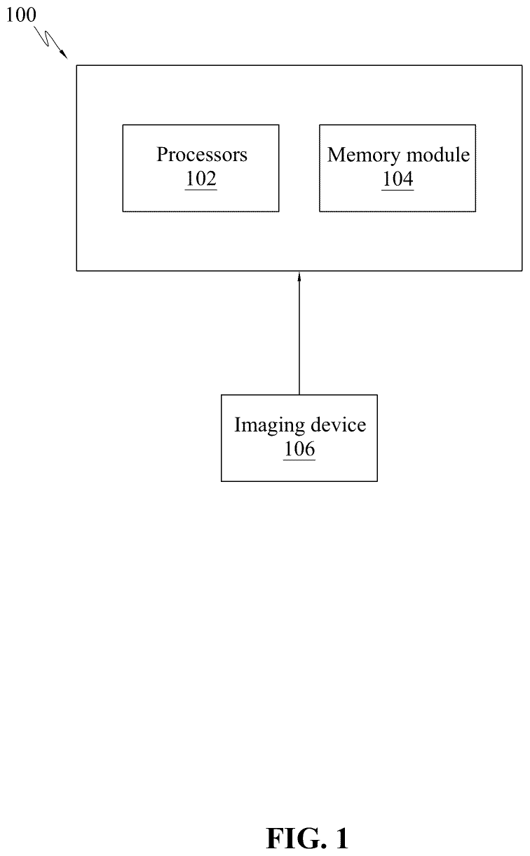

FIG. 1 illustrates a block diagram of a system for anonymizing videos, wherein the system may be in communication with an imaging device 106. The system comprises of a server 100 comprising of one or more processors 102 and a memory module 104.

In an embodiment, the one or more processors 102 may be implemented as appropriate in hardware, computer-executable instructions, firmware, or combinations thereof. Computer-executable instruction or firmware implementations of the one or more processors 102 may include computer-executable or machine-executable instructions written in any suitable programming language to perform the various functions described.

In an embodiment, the memory module 104 may include a permanent memory such as hard disk drive, may be configured to store data and executable program instructions that are implemented by the one or more processors 102. The memory module 104 may be implemented in the form of a primary and a secondary memory. The memory module 104 may store additional data and program instructions that are loadable and executable on the one or more processors 102, as well as data generated during the execution of these programs. Further, the memory module 104 may be a volatile memory, such as random-access memory and/or a disk drive, or a non-volatile memory. The memory module 104 may comprise of removable memory such as a Compact Flash card, Memory Stick, Smart Media, Multimedia Card, Secure Digital memory, or any other memory storage that exists currently or may exist in the future.

In an embodiment, the imaging device 106 may not be an integral part of the system. The imaging device 106 may be, but not limited to, camera glasses, head mounted cameras, augmented/mixed reality glasses, any mechanical, digital, or electronic viewing device, still camera, camcorder, motion picture camera, or any other instrument or equipment capable of recording, storing, or transmitting visual images.

The video captured by the imaging device 106 may comprise of a real-time video streaming and/or offline and/or live streamed videos. Each of the videos captured may comprise of a plurality of video frames, wherein each video frame comprises of a plurality of pixels.

In another embodiment, imaging device may form an integral part of the system. For example, a cell phone with sufficient processing power, wherein the cell phone is provided with a camera.

In an embodiment, the system may be provided with additional components in addition to the ones discussed in the foregoing. The additional components may be decided based on the requirements.

In an embodiment, the server 100 may be cloud based, wherein all the data processing is performed external to any specific device. The cloud-based server 100 may offer better performance and flexibility as it is not restricted to any physical device thereby users can utilize the server 100 without having to be in a specific location. In such cases the imaging device 106 and the server 100 may be configured to be in communication over a communication network, wherein the communication network may be, but not limited to, a local network, wide area network, a metropolitan area network or any wireless network.

In another embodiment, server 100 comprising of one or more processors 102 and memory module 104 may be an integral part of any computing device such as, but not limited to, desktop, laptop, cell phones, tablets and augmented reality device.

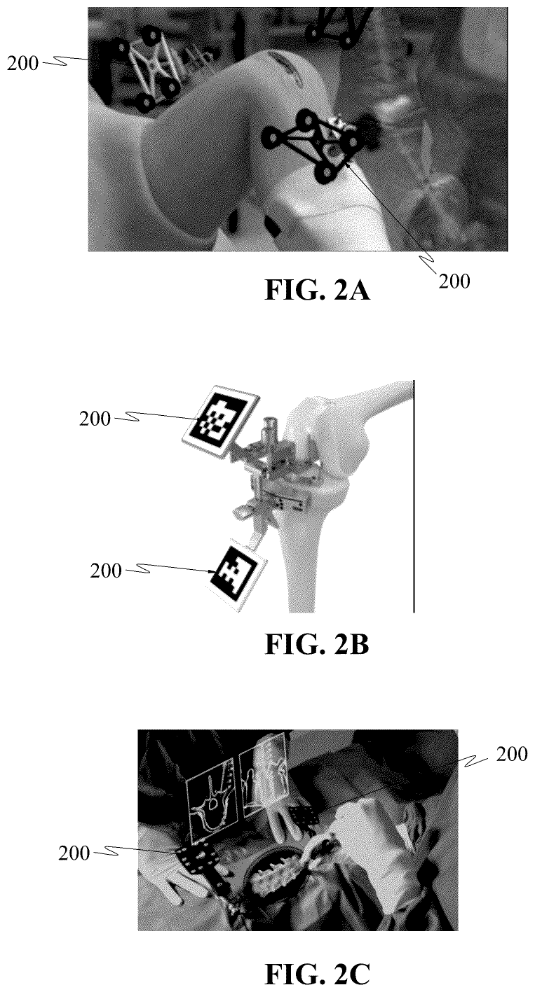

Referring to FIGS. 2A-2C, one or more reference entities 200 may be employed, wherein the reference entities 200 may be configured to be disposed on various surfaces. The reference entities 200 may be employed to determine a Region of Interest (ROI). Video frame with one or more reference entities 200 may be predicted to have one or more ROI, while video frame without reference entities 200 may be predicted to comprise sensitive information.

In an embodiment, the reference entities 200 may be, but not limited to, physical markers or flags, proxy indicators, fiducial markers, AR markers (Augmented Reality markers), bone markers or patterns (similar to a barcode, a quick response code (QR code), augmented reality fiducial marker etc.). The reference entities 200 may be configured to be made detectable by the one or more processors 102 of the server 100. The reference entities 200 may be configured to be applied over any type of surface.

In an embodiment, the one or more processors 102 may be configured to detect presence or an absence of one or more reference entities 200 in a video or an image. The one or more processors 102 may be trained to detect one or more reference entities 200. The one or more processors 102 may also be trained to detect different objects in addition to detecting reference entities 200. When any reference entities 200 are captured in a video or an image by the imaging device 106, the one or more processors 102 may be configured to detect the one or more reference entities 200 in the captured plurality of video frame of a video or the image.

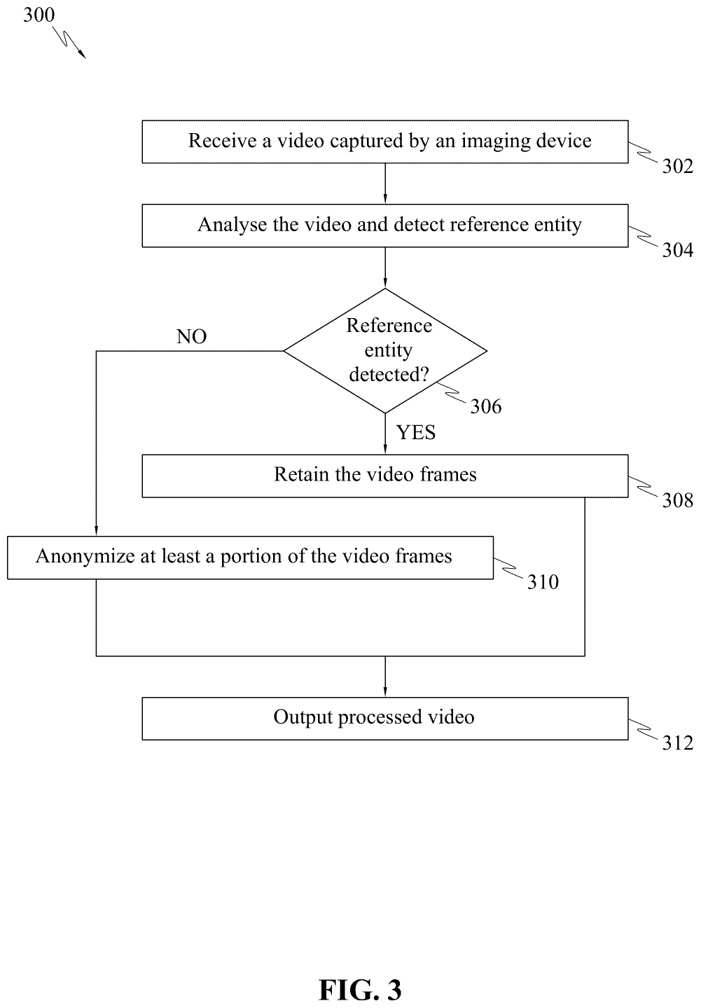

FIG. 3 illustrates a flow chart 300 depicting a method of an embodiment for anonymizing videos employing the system, in accordance with an embodiment.

At step 302, the system may be configured to receive a video captured by the imaging device 106. The video may be, but not limited to, a real-time video and/or offline and/or live streamed video.

At step 304, the one or more processors 102 of the system may be configured to analyse each video frames among the plurality of video frames and detect presence or absence of one or more reference entities 200 in each video frame.

At step 306, the one or more processors 102 of the system may be configured to determine presence or absence of one or more reference entities 200 in each video frame.

At step 308, if one or more reference entities 200 are detected, the one or more processors 102 of the system may be configured to retain the video frames. For example, when the one or more processors 102 detect one or more reference entities 200 in a video frame, the one or more processors 102 are configured to retain the original video frame without altering/editing the video frame as the one or more processors 102 detect a ROI.

At step 310, if one or more reference entities 200 are not detected, the one or more processors 102 of the system may be configured to anonymize at least a portion of the video frames. For example, when the one or more processors 102 fail to detect one or more reference entities 200 in a video frame, the one or more processors 102 are configured to alter/edit the video frame by anonymizing at least a portion of the video frame or the entire video frame as no ROI is detected. Altering/editing of the video frame comprises of anonymizing portions of video frames or deleting the entire video frame.

Anonymize may include protection of sensitive information by obscuring, removing, preventing capture, preventing storage, or otherwise preventing unwanted use of such information.

At step 312, the one or more processors 102 may be configured to output a processed video comprising of plurality of anonymized and original video frames, which can then be used as per requirements.

The processed video may be stored for future use or may be live streamed to an interested audience.

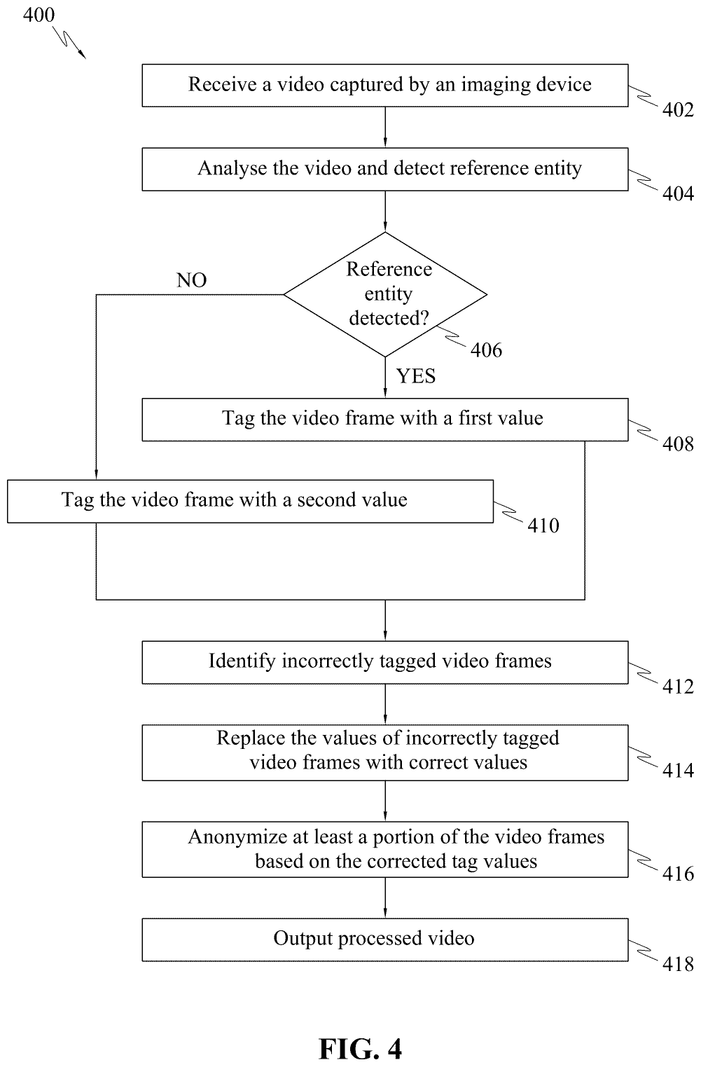

FIG. 4 illustrates a flow chart 400 depicting a method of another embodiment for anonymizing videos by tagging video frames, in accordance with an embodiment.

At step 402, the system may be configured to receive a video captured by the imaging device 106. The video may be, but not limited to, a real-time video and/or offline and/or live streamed video.

At step 404, the one or more processors 102 of the system may be configured to analyse each video frames among the plurality of video frames and detect presence or absence of one or more reference entities 200 in each video frame.

At step 406, the one or more processors 102 of the system may be configured to determine presence or absence of one or more reference entities 200 in each video frame.

At step 408, if the one or more processors 102 detect one or more reference entities 200 in a video frame, the one or more processors 102 may be configured to tag that particular video frame with a first value. For example, when the one or more processors 102 detect one or more reference entities 200 in a video frame, the one or more processors 102 may be configured to tag that particular video frame with the first value i.e., “0”.

At step 410, if the one or more processors 102 fail to detect one or more reference entities 200 in a video frame, the one or more processors 102 may be configured to tag that particular video frame with a second value. For example, when the one or more processors 102 fail to detect one or more reference entities 200 in a video frame, the one or more processors 102 may be configured to tag that particular video frame with the second value i.e., “1”.

The first value and the second value may enable the one or more processors 102 to identify and differentiate the video frames into two categories i.e., video frames comprising one or more reference entities 200, and video frames without any reference entities 200. This helps in further processing of the video by the one or more processors 102.

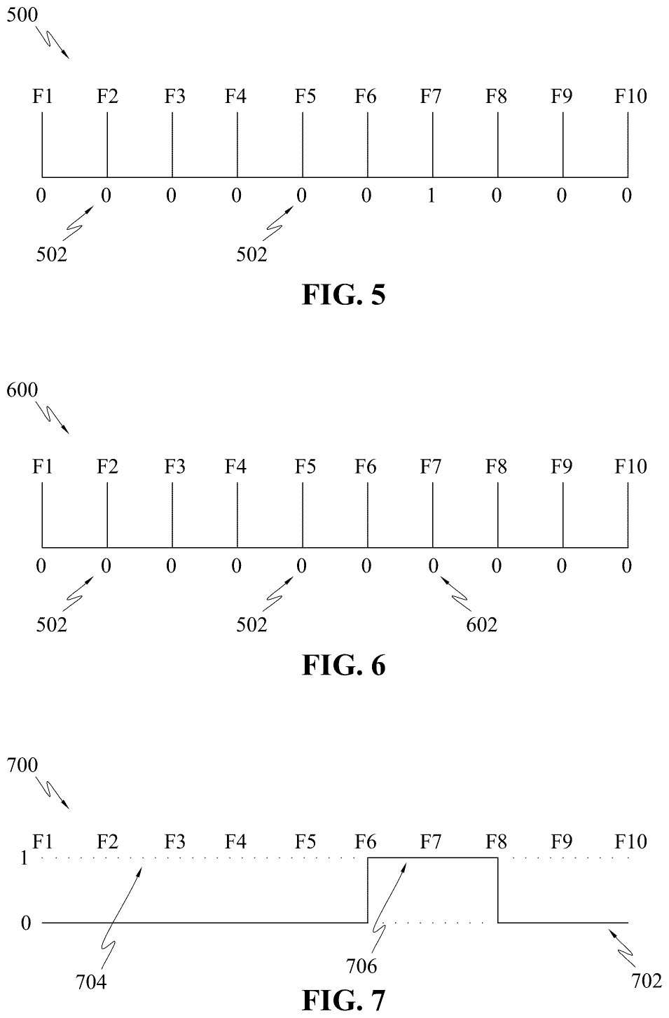

At step 412, the one or more processors 102 may be configured to identify incorrectly tagged video frames among the plurality of video frames by analysing tagged values associated with immediately preceding and following video frames. The incorrectly tagged video frame is identified by inspecting the tagged values associated with a set of consecutive preceding and following video frames, wherein a video frame is confirmed to be incorrectly tagged when tagged value of the video frame is different from the tagged values of the set of consecutive preceding and following video frames. For example, when the one or more processors 102 of the system are provided with a plurality of tagged video frames, the one or more processors 102 are configured to identify incorrectly tagged video frame(s) among the plurality of video frames by inspecting the tagged values of a set of consecutive preceding and following video frames. As an example, for a video comprising 500 video frames, a set of 10 consecutive preceding and following video frames from a video frame being analysed may be inspected in each inspection cycle. The values for a set may be determined based on the number of video frames being analysed and may not be fixed. The one or more processors 102 determines that a particular video frame is incorrectly tagged when tagged value of that particular video frame is different from the tagged values of the set of consecutive preceding and following video frames. Referring to FIG. 5, a video 500 comprising of 10 video frames (F1-F10) is depicted. Each video frame is tagged 502 with the first value “0” or the second value “1”. In this example, video frame F7 is identified to be incorrectly tagged as set of three consecutive preceding and following video frames have tag values different from that of the video frame F7.

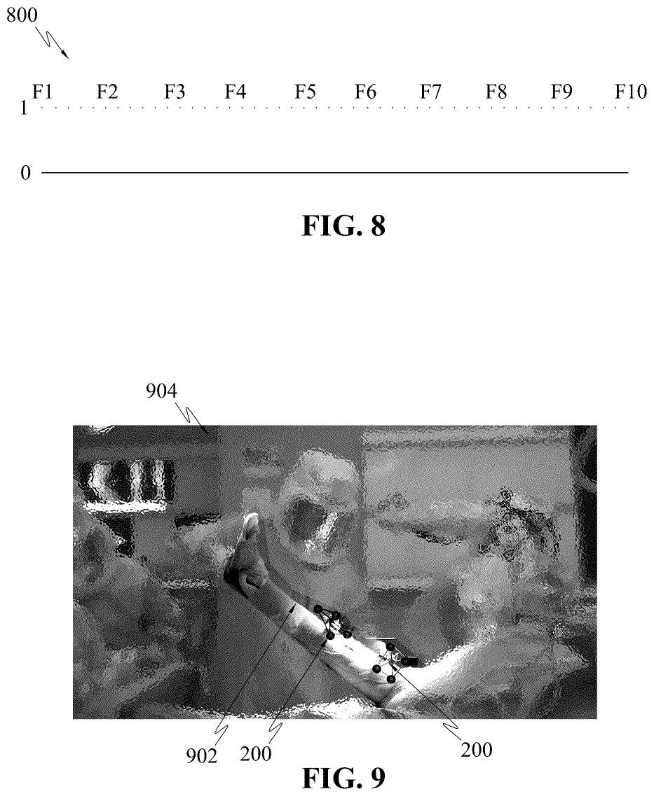

At step 414, the one or more processors 102 may be configured to replace the value of the incorrectly tagged video frames with a value of one of the preceding or following video frames. The tagged value of the identified incorrectly tagged video frame is replaced with a value of at least one of the tagged video frames among the inspected set of consecutive preceding and following video frame. For example, referring to FIG. 6 a video 600 comprising of 10 video frames (F1-F10) with the corrected tag values is depicted. The one or more processors 102 are configured to identify that the incorrectly tagged video frame F7 (refer FIGS. 5-6) is a false positive/false trigger and thereby replace the value of the incorrectly tagged video frame F7 with a corrected tag value 602, wherein the corrected tag value is a tag value of one video frame among the inspected set of consecutive preceding and following video frame (which is “0” in case of FIGS. 5-6). Also refer to FIGS. 7-8, wherein a video is represented by way of graph, wherein a lower line 702 represents the first value “0” and an upper line 704 represents the second value “1”, wherein FIG. 7 represents video comprising incorrectly tagged portions 706 and FIG. 8 represents video with the corrected tag values.

At step 416, the one or more processors 102 may be configured to anonymize based on the updated tagged values of the video frames, wherein the updated values may either be “0” or “1”. If the tagged value of video frames is 0, the one or more processors 102 of the system may be configured to retain the video frame. If the tagged value of video frames is 1, the one or more processors 102 of the system may be configured to anonymize at least a portion of the video frames, the complete video frame or delete the video frame.

At step 418, the one or more processors 102 may be configured to output a processed video comprising of plurality of anonymized and original video frames, which can then be used as per requirements.

In an embodiment, the one or more processors 102 may be configured to analyse whole video comprising of plurality of video frames for tagging the video frames with respective first value or second value. The one or more processors 102 may also be configured to break down the video into multiple sets comprising of plurality of video frames, wherein each set is then analysed independently by the one or more processors 102.

In an embodiment, the one or more processors 102 may be configured to predict orientation of one or more reference entities 200 in each of the video frames. The one or more processors 102 may be further configured to determine position of one or more reference entities 200 in each of the video frames. The one or more processors 102 may also be configured to determine size of one or more reference entities 200 in each of the video frames.

In an embodiment, the one or more processors 102 may be configured to determine the position of one or more reference entities 200 by, but not limited to, mapping a Coordinate System (CS) onto the reference entities 200 in the video frames. When a reference entity 200 is detected on the video frame, the coordinate system of the reference entity 200 enables calculation of its position and orientation in three-dimensional space relative to the camera, for e.g. in terms of rotational and translational units in cartesian X-axis, Y-axis and Z-axis.

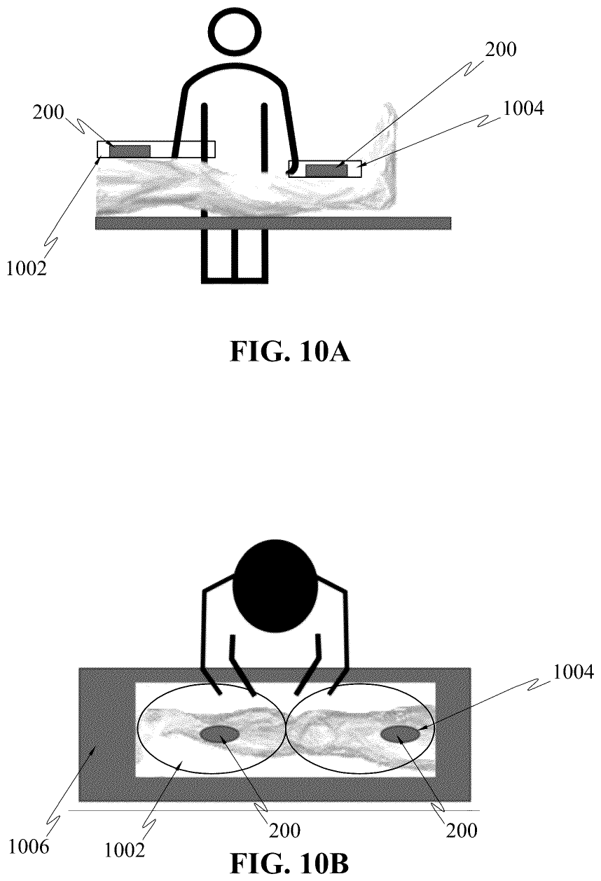

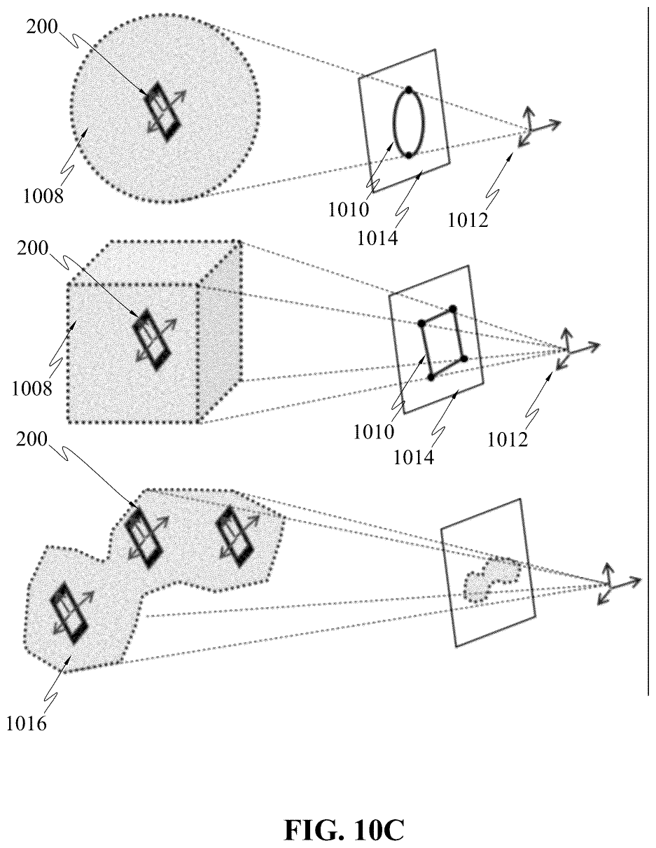

Referring to FIG. 10C, once a reference entity 200 is detected, and its orientation relative to camera CS 1012 is predicted, the one or more processors 102 may use the predicted orientation to create a pre-defined 3D volume 1008 around the reference entity 200 to determine its projection (2D ROI) 1010 on the camera image 1014. The pre-defined 3D volume 1008 may be of any shape, and of non-uniform extent in different directions. Pixels/portions of the video frame/image outside this ROI 1008 may be then marked for anonymization.

In another embodiment, one or more reference entities 200 may also be used in groups to generate uneven shapes/volumes to envelope any ROI 1016 to generate an, but not limited to, an oval volume to capture the full ROI. Complex 3D volumes of any shape, including for example 3D models of bones created from a medical scan can be mapped to the reference entity CS and used to create a ROI closely matching a patient's anatomy. As the reference entity 200 moves relative to camera, the corresponding projected 2D ROI also changes continuously.

In an embodiment, the one or more processors 102 may be configured to determine the size of the reference entity 200, by referring to a reference size of an image of the reference entity stored in the memory module 104, wherein the reference size of the reference entity image is determined by taking an image of the reference entity 200 from a predetermined distance. If one or more reference entities 200 in the video frames are smaller than the reference size, the one or more processors 102 may predict that the one or more reference entities 200 are farther than the stored “predetermined distance”, thereby predicting that the video frame has a wider field of view. If one or more reference entities 200 in the video frames are bigger than the reference size, the one or more processors 102 may predict that the one or more reference entities 200 are in proximity thereby predicting that the video frame has a narrower field of view.

In an embodiment, the one or more processors 102 may be configured to predict a point of view based on the detected orientation of the reference entity 200.

In an embodiment, the one or more processors 102 may be configured to predict field of view captured in each of the video frames based on the determined size and the position of the reference entity 200 in each video frame.

In an embodiment, the one or more processors 102 may be configured to identify portion of video frames with one or more reference entities 200 as ROI 902 (refer FIG. 9). Based on the identified ROI 902, the one or more processors 102 may be configured to anonymize portions of video frames 904 other than ROI 902. Anonymization of the video frames may also be determined based on the predicted point of view and field of view, wherein portions of video frames or certain pixels of the video frames may be anonymized based on predetermined points of view or fields of view, wherein different points of view or fields of view may be anonymized differently (which can be preconfigured).

For example, when one or more video frames comprises of one or more reference entities 200, the one or more processors 102 are configured to use the position of the one or more reference entities 200 to define a second portion 1002 or a fourth portion 1004. The one or more processors 102 are configured to then predict point of view and field of view. The one or more processors 102 may be configured to, based on the predicted point of view and field of view, anonymize portions 1006 of the video frames other than the second portion 1002 or the fourth portion 1004 or anonymize complete video frame, by referring to the preconfigured point of view and field of view. As an example, referring to FIG. 10A, based on the position and orientation of the one or more reference entities 200, the one or more processors 102 may predict that the point of view is a side view. When the one or more processors 102 predicts a side view, the entire video frame may be anonymized. In another example, referring to FIG. 10B, based on the position and orientation of the one or more reference entities 200, the one or more processors 102 may predict that the point of view is a top view. When the one or more processors 102 predicts a top view, only portions of the video frame other than the second portion 1002 or the fourth portion 1004 are anonymized.

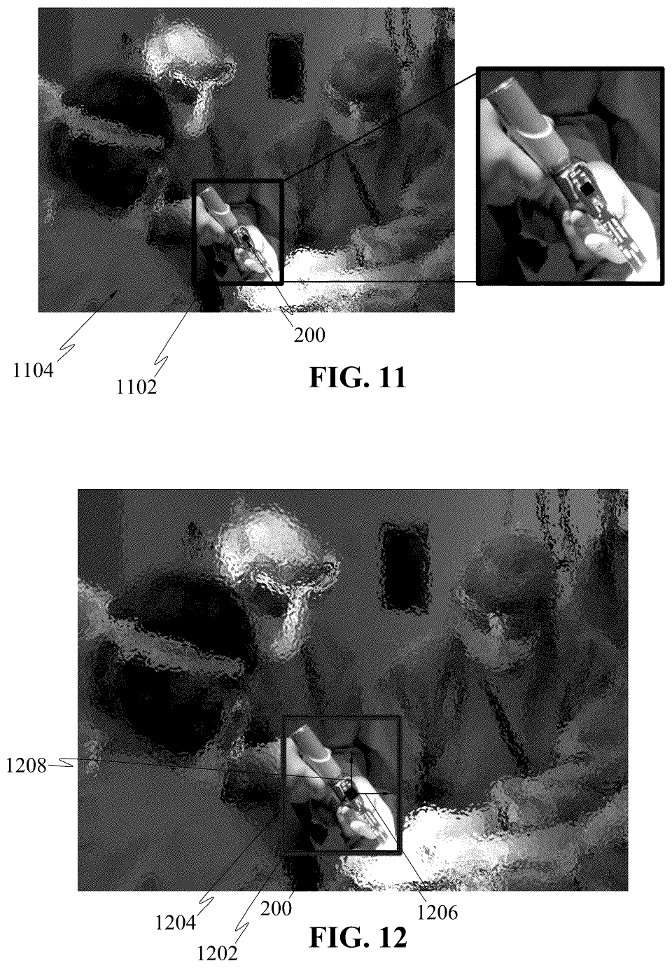

Referring to FIG. 11, when one or more video frames comprises of one or more reference entities 200, the one or more processors 102 are configured to use the position of the one or more reference entities 200 to define a third portion 1102 or a fifth portion 1102. The one or more processors 102 are configured to then predict point of view and field of view. The one or more processors 102 are configured to, based on the predicted point of view and field of view, zoom into at least the third portion 1102 or the fifth portion 1102 of video frames and anonymize portions of the video frames 1104 other than the third portion 1102 or the fifth portion 1104, by referring to the preconfigured predetermined point of view and field of view.

In an embodiment, third portion and fifth portion may be two different portions on video frame.

Referring to the same figures, FIGS. 10A-10B, in another embodiment, the one or more processors 102 may be configured to anonymize video frames based on shape of one or more reference entities 200 detected in the video frames. For example, if one or more reference entities 200 are detected as oval shapes, the one or more processors 102 may be configured to predict the point of view as a top view. And, if one or more reference entities 200 are detected as rectangle shapes, the one or more processors 102 may be configured to predict the point of view as a side view.

In another embodiment, the one or more processors 102 may be configured to anonymize entire video frames for a predetermined field of view and a predetermined point of view.

Referring to FIG. 12, in an embodiment, when one or more video frames comprises of one or more reference entities 200, the one or more processors 102 may be configured to determine position of the one or more reference entities 200 as a sixth portion 1202. The one or more processors 102 may be configured to then predict point of view and field of view. The one or more processors 102 may then be configured to, based on the predicted point of view and field of view, anonymize portions of the video frames other than a predetermined area 1204, wherein the predetermined area may be calculated by taking the reference entity 200 as a reference point. The predetermined area may be calculated in both X-axis 1206 and Y-axis 1208 of the video frames.

In an embodiment, the predetermined area may be in a form of circle, ellipse, rectangle and any polygon, among others, wherein the one or more processors 102 may be configured to not anonymize the predetermined areas 1204 thereby retaining the ROI, while rest of the portions of the video frames are anonymized. The predetermined area 1204 may be customized for different points of view or fields of view.

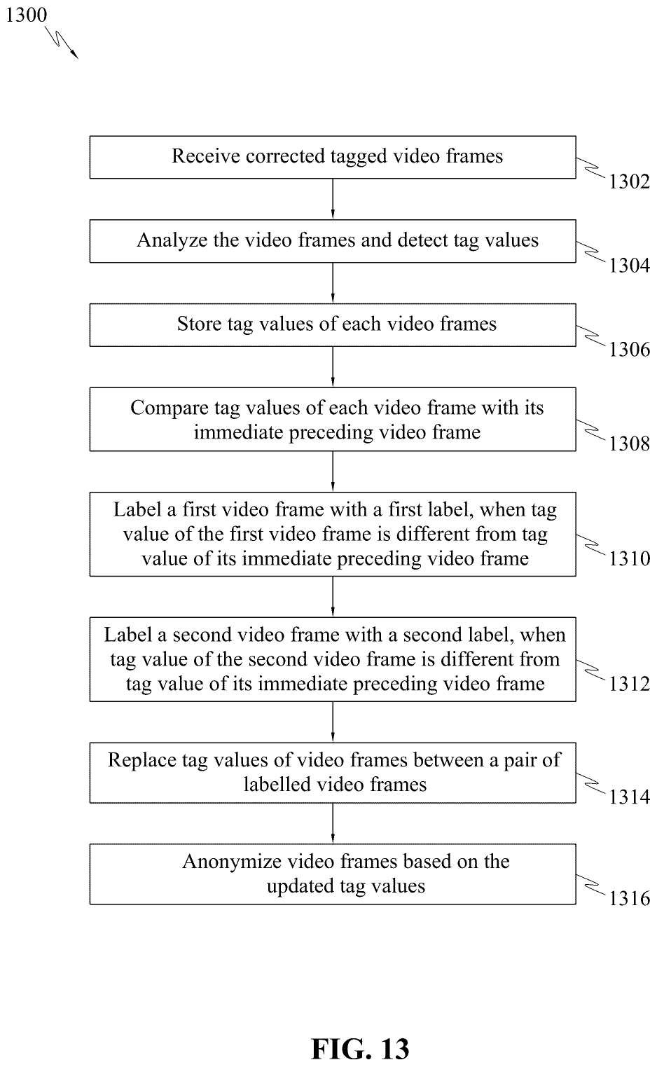

It may be noted that tagging and tag correction has an important role in anonymizing the video frames. We will now discuss an additional embodiment corresponding to tagging. Continuing from the previously discussed tag correction (step 414 of FIG. 4 discussed previously), after the tagged values of the incorrectly tagged video frames are corrected, the corrected tagged video frames may be reanalysed by a tag filtering process. FIG. 13 illustrates a flowchart 1300 depicting the tag filtering process.

At step 1302, one or more processors 102 of the system may be configured to receive the corrected tagged video frames.

At step 1304, the one or more processors 102 of the system may be configured to analyse each video frames and detect values tagged for each video frame.

At step 1306, the one or more processors 102 of the system may be configured to store the tag value of each video frame.

At step 1308, the one or more processors 102 of the system may be configured to compare tag value of one of the video frames with tag value of its immediately preceding video frame.

At step 1310, the one or more processors 102 may be configured to label a first video frame with a first label, when tag value of the first video frame is different from tag value of its immediately preceding video frame, wherein the first label indicates occurrence of a change in tag value.

At step 1312, the one or more processors 102 may be configured to label a second video frame with a second label, when tag value of the second video frame is different from tag value of its immediately preceding video frame, wherein the second label indicates occurrence of a change in tag value.

At step 1314, the one or more processors 102 may be configured to replace all tag values of video frames between the video frame with the first label and the video frame with the second label, wherein the tag values may be replaced with tag value of the frame immediately preceding the frame with the first label.

At step 1316, the one or more processors 102 may be configured to anonymize video frames based on the updated tag values of the video frames.

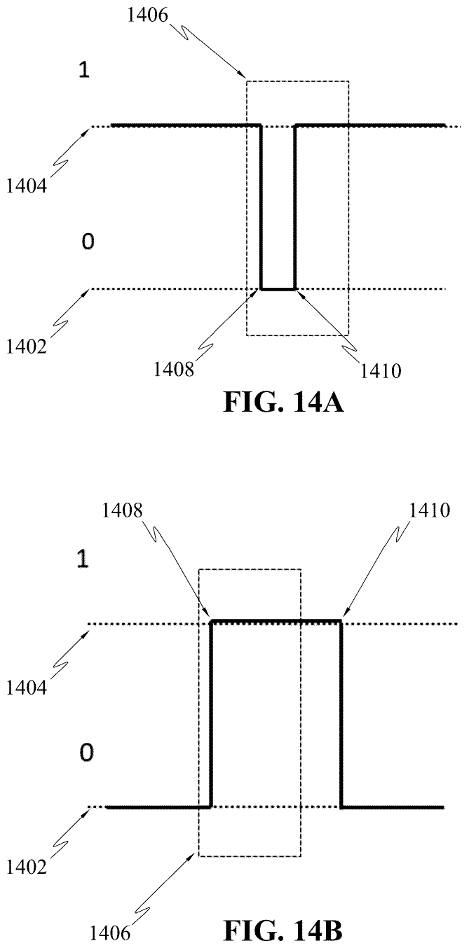

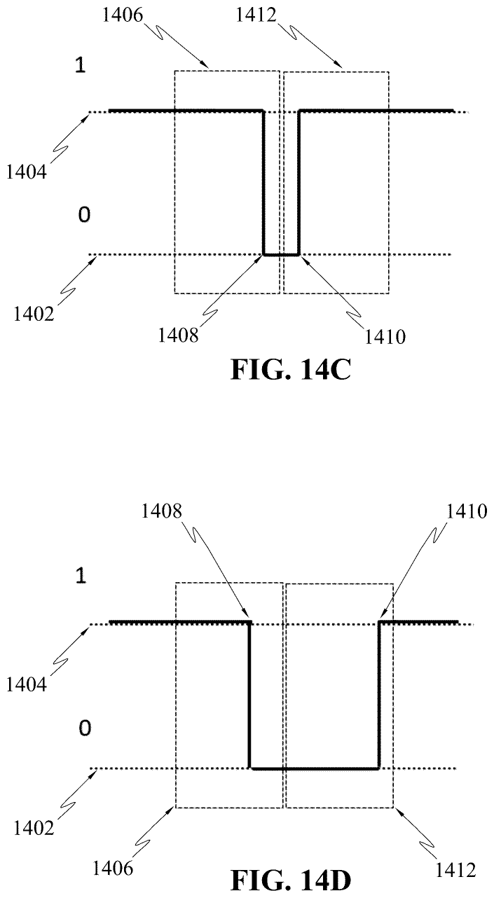

An example explaining the above process is now described in greater detail (refer FIGS. 14A-14D). The one or more processors 102 may define a number of video frames to be considered in each set (W) for analysing a video, wherein each video frame is tagged with a first value 1402 or a second value 1404 based on the presence or absence of reference entity 200. Let's say number of video frames are defined as, but not limited to, 20 i.e., W=20, then W+2 i.e., 22 video frames are considered by the one or more processors 102 for analysis. The video comprising of plurality of video frames are now divided into multiple sets comprising of 22 video frames which are considered by the one or more processors 102 for analysis. The last set of video frames may comprise all remaining frames<W+2.

Upon creating multiple sets of video frames, each set is analysed, wherein tagged values of video frames in a first set of video frames 1406 is detected by the one or more processors 102 and stored in a vector “T” for each video frame, wherein each frame is labelled T[i], wherein “i” represents the video frame and “T[i]” represents the tag value of that video frame. Therefore, a second video frame having a tagged value “0” is represented as T[2]=0.

Once the tagged values of all the video frames from the first set of video frame 1406 are stored, tagged value of each video frame in the first set of video frames 1406 starting from second video frame among the plurality of video frame in the first set of video frames 1406 is compared with tagged value of its immediate preceding video frame in the same first set of video frames 1406 i.e., the one or more processors are configured to compare the values of T[i] and T[i−1] i.e., comparing tagged values of T[2] and T[1] and so on.

The one or more processors 102 are configured to label a video frame with a first label 1408, if tagged value of T[i] and T[i−1] are different. The frame number (i) with first label 1408 is stored in a vector “change”, i.e. change[j]=i, representing the location of a change in the tagged values between two adjacent video frames.

Similarly, the one or more processors 102 are configured to label a video frame with a second label 1410 or a third label and so on, if tagged value of any T[i] and T[i−1] are different. The frame number with second label 1410 is stored in the next element (j+1) of vector change (i.e change[j+1]), wherein tag value of change[j+1] is the tag value of respective video frame (T[change (j+1)]), and the frame number with third label is stored as change[j+2], wherein tag value of change[j+2] is the tag value of respective video frame ((T[change (j+2)]) and so on, wherein the second label 1410 and third label represent change in the tagged values between two adjacent video frames. The second label 1410, the third label and so on are assigned to any subsequent video frames where tagged values of two adjacent video frames are different, wherein adjacent video frames implies a video frame and its immediately preceding video frame.

Once the video frames in the first set of video frames 1406 are labelled, the one or more processors 102 are configured to pair the labelled video frames and then change the tagged values of the video frames that lie between pair of the labelled video frames. For example, the video frame labelled with the first label 1408 and the video frame labelled with the second label 1410) are replaced with tag value of the frame immediately preceding the frame with the first label.

Let's say, in the first set of video frames 1406, the first change in tag value is detected at a third frame, wherein the tag value of the second video frame is “0” and tag value of the third video frame is “1”, the one or more processors 102 then labels the third frame with the first label 1408 and stores this frame number in “change”, i.e. change[1] (change[j])=3. Tag value of change[1], i.e. T[change[1]]=1. A second change in the tag value if detected at fifth frame, wherein the tag value of the fourth video frame is “1” and tag value of the fifth video frame is “0”, the one or more processors 102 then labels the fifth frame with the second label 1410 i.e., change[2] (change[j+1])=5. Tag value of change[2], i.e. T[change[2]]=0.

Now the one or more processors 102 are configured to pair the labelled video frames i.e., change[1] 1408 and change[2] 1410 and then replace tagged values of each of the video frames between the pair of labelled video frames i.e., change[1] 1408 and change[2] 1410 with the tag value of frame preceding the frame labelled with first label i.e., [T[change[1]−1]=T[2]=0, wherein the tag values are now replaced with the tag value “0”.

In an embodiment, the one or more processors 102 may be configured to take into account a variety of input variables including, frame rate of a video, video resolution, type/context of video (e.g. video captured in an operating room, video captured during a physical therapy session etc.). For example, in videos captured at a high frame rate, W≤20 frames maybe considered a false prediction, while for videos captured at a slower frame rate, W≤2 frames can be considered a false prediction. Similarly, lower video resolution may make prediction task more challenging than higher video resolution, and therefore definition/treatment of value W could be varied based on video resolution.

In yet another embodiment, user maybe provided with the ability to control the “sensitivity” of the tag pattern analysis, such that the algorithm can be biased to a lesser or greater extent towards anonymizing a given frame when the certainty of the original tag/prediction is below a pre-set threshold.

In another embodiment, all or a subset of frames from the set W maybe presented to the user to get his/her inputs to guide the anonymization process.

In another embodiment, tag filtering process may be implemented for videos that are retrieved from a database, wherein the videos are not analysed previously, or for videos that are fed directly from the imaging device 106 (online streaming), without having to analyse the video as discussed previously in FIG. 4. However, in cases where videos are directly analysed as discussed in FIG. 13, each video needs to be analysed to detect one or more reference entities 200 and tag each video frame with either first value or second value and then proceed with the process as described in FIG. 13.

In cases where the one or more processors 102 fail to pair the labelled video frames within the first set 1406 of video frames, the one or more processors 102 are configured to pair the last labelled video frame in the first set 1406 of video frames with a labelled video frame in the second set 1412 of video frames if the difference in the two frame numbers is <=W (Refer FIG. 14C where such pairing is performed, while in example of FIG. 14D such pairing is not performed). In general, the above-described tag filtering process can remove any abrupt changes in tag values spanning <=W frames.

The tag filtering process may be employed to address the challenges of false predictions and help to reduce instances where either a frame containing sensitive information does not get anonymized, or a frame that does not contain sensitive information gets anonymized, leading to loss of information that may have had utility for the end use case.

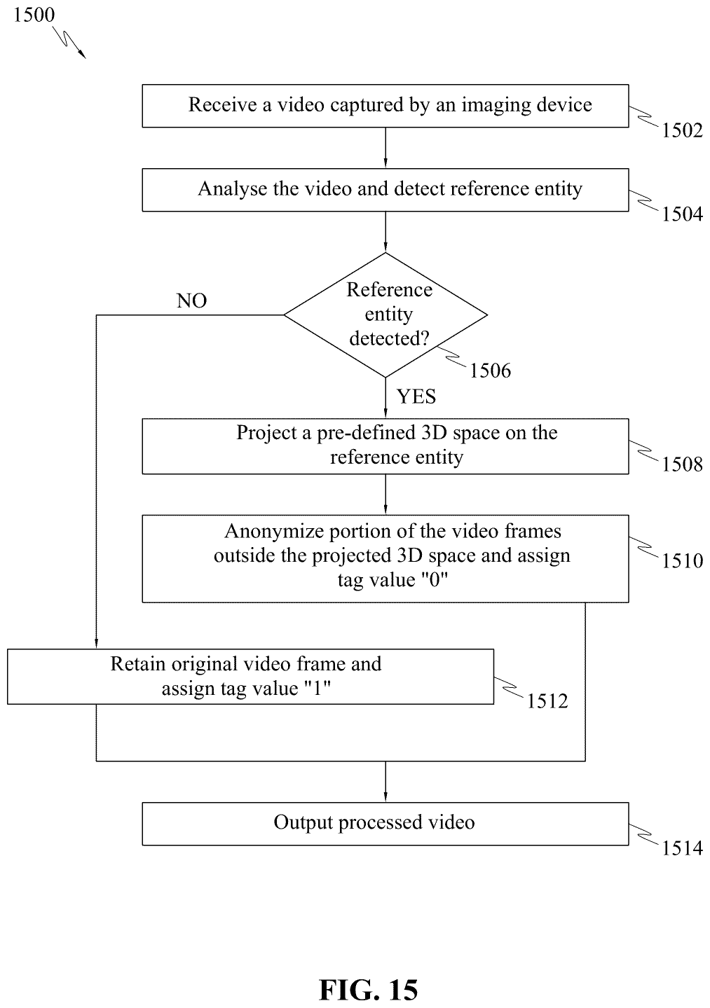

FIG. 15 illustrates a flowchart 1500 of a method of another embodiment for anonymizing videos. In this embodiment, the system may be configured to project a 3D space/volume around or adjacent to locations of detected reference entities 200.

At step 1502, the system may be configured to receive a video captured by the imaging device 106. The video may be, but not limited to, a real-time video and/or offline and/or live streamed video.

At step 1504, the one or more processors 102 of the system may be configured to analyse each video frames among the plurality of video frames and detect presence or absence of one or more reference entities 200 in each video frame.

At step 1506, the one or more processors 102 of the system may be configured to determine presence or absence of one or more reference entities 200 in each video frame.

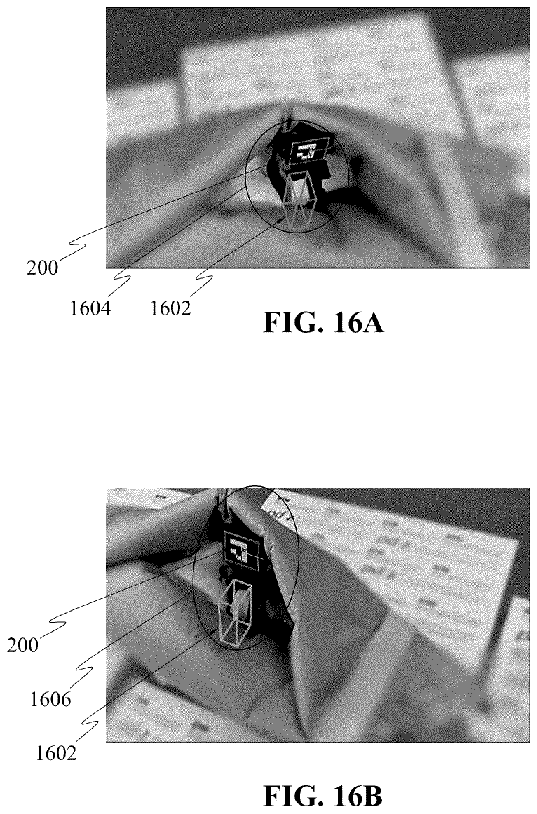

At step 1508, if one or more reference entities 200 are detected, the one or more processors 102 of the system may be configured to project a pre-defined 3D space 1008 (as explained in the foregoing description) onto portions of the video frames with the reference entities 200 (refer FIGS. 16A-16B). For example, an augmented reality fiducial marker (an AR tag), may be used to calculate the reference entity's relative position and orientation relative to the imaging device 106 (camera) i.e. the reference entity coordinate system (CS). A 3D cuboid (1602) that tracks together with the reference entity CS and shows how the cuboid's appearance in the video frames/camera image changes based on viewing direction. Any of several known methods for calculating AR tag relative position and orientation may be used.

At step 1510, the one or more processors 102 of the system may be configured to anonymize portions of the video frames outside the predicted ROI and assign a first tag value “0”. Anonymization may be determined based on the size of the projected 3D space (3D space in turn defines the ROI), wherein small 3D space 1008 may correspond to smaller ROI 1604 and thus anonymization of larger portions of the video frames and bigger 3D space 1008 may correspond to bigger ROI 1606 and thus anonymization of smaller portions of the video frames (refer FIGS. 16A-16B).

At step 1512, if one or more reference entities 200 are not detected, the one or more processors 102 of the system may be configured to retain the original video frames and assign a second tag value “1”.

At step 1514, the one or more processors 102 may be configured to output the processed video along with tag values assigned to each of the video frames.

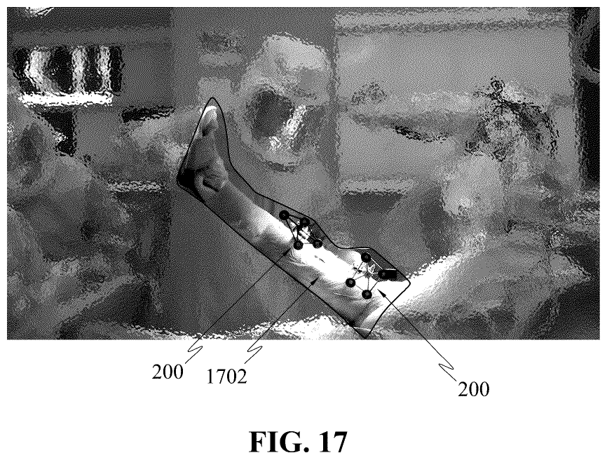

Referring to FIG. 17, when the one or more processors 102 detect one or more reference entities 200 in proximity, the one or more processors 102 may be configured to create a virtual area 1702 around the detected plurality of reference entities 200 and anonymize portions of the video frames other than the virtual area 1702.

In an embodiment, the virtual area 1702 may be determined based on position and orientation of the reference entities 200. The virtual area 1702 may be configured to cover portions of the plurality of reference entities 200 along with portions disposed between two or more reference entities 200. Complex 3D volumes of any shape (Refer FIG. 10C), including for example 3D models of bones created from medical scans, can be mapped to the coordinate system of the reference entity to create a 3D ROI closely matching the patient's anatomy. As the marker moves relative to camera, the corresponding projected 2D ROI also changes continuously.

In an alternate embodiment, the system, in addition to being trained to detect one or more reference entities 200, may also be trained to identify video frames or a specific portion of the video frames comprising sensitive information for anonymizing videos or images, wherein sensitive information comprises of, but not limited to, faces, portions of faces, computer screens containing information, x-ray images on screens and documents visible in the video.

In yet another embodiment, the system may be trained to anonymize portions of video frames or images where one or more reference entities 200 are present, while rest of the portions of the video frames or images are not anonymized. With this approach, reference entities 200 may be disposed on various locations such as, but not limited to, computer screens in an Operation Theatre, any portion of human face and certain body parts of patient, doctor or health care assistants, that are needed to be labelled as sensitive information. Similar process of tagging the video frames and correcting the tagged video frames along with tag filtering process may be employed for an optimum output.

In an embodiment, the system configuration may be employed in non-medical applications such as, but not limited to, retail spaces, event spaces, shopping malls, space research centres, labs, wherein the reference entities 200 may be disposed on, but not limited to, a person, shelf in a lab containing sensitive data or screens in a research centre. The system may be configured to anonymize portions of video frames comprising of reference entities 200.

In yet another embodiment, system may be trained by providing manually labelled video frames, wherein faces of surgical staff (who typically are wearing masks, face shields, head covers etc.), hands, instruments, and screens were demarcated with bounding boxes and assigned corresponding labels (face, instrument, hand or screen). Anonymization may be done based on the detected bounding boxes.

The processes described above is described as a sequence of steps, this was done solely for the sake of illustration. Accordingly, it is contemplated that some steps may be added, some steps may be omitted, the order of the steps may be re-arranged, or some steps may be performed simultaneously.

Although embodiments have been described with reference to specific example embodiments, it will be evident that various modifications and changes may be made to these embodiments without departing from the broader spirit and scope of the system and process or method described herein. Accordingly, the specification and drawings are to be regarded in an illustrative rather than a restrictive sense.

Many alterations and modifications of the present invention will no doubt become apparent to a person of ordinary skill in the art after having read the foregoing description. It is to be understood that the phraseology or terminology employed herein is for the purpose of description and not of limitation. It is to be understood that the description above contains many specifications, these should not be construed as limiting the scope of the invention but as merely providing illustrations of some of the personally preferred embodiments of this invention.

What is claimed is:

1. A system for anonymizing videos, the system comprising:
a server, wherein the server comprises one or more processors configured to:
receive a video captured by an imaging device, wherein the video comprises of a plurality of video frames;
analyse each of the video frames captured by the imaging device and detect reference entity in each of the video frames; and
anonymize at least a portion of each of the video frames in which the reference entity is absent.

2. The system according to claim 1, wherein the reference entity is a fiducial marker configured to be detected by the one or more processors of the server.

3. The system according to claim 1, wherein the one or more processors are configured to:
tag a video frame with a first value if one or more reference entity is detected in the video frame; and
tag a video frame with a second value if reference entity is absent in the video frame.

4. The system according to claim 3, wherein the one or more processors are configured to:
identify incorrectly tagged video frames among the plurality of video frames by analysing tagged values of immediately preceding and following video frames; and
replace the value of the incorrectly tagged video frames with a value of one of the preceding or following tagged video frames.

5. The system according to claim 4, wherein:
the incorrectly tagged video frame is determined by inspecting the tagged values of a set of consecutive preceding and following video frames, wherein a video frame is confirmed to be incorrectly tagged when tagged value of the video frame is different from the tagged values of the set of consecutive preceding and following video frames; and
the tagged value of the determined incorrectly tagged video frame is replaced with the value of at least one of the video frames among the inspected set of consecutive preceding and following video frame.

6. The system according to claim 1, wherein the one or more processors are configured to:
predict orientation of the reference entity in each of the video frames;
predict position of the reference entity in each of the video frames; and
predict size of the reference entity in each of the video frames.

7. The system according to claim 6, wherein the one or more processors are configured to:
predict point of view of the reference entity in each of the video frames based on the detected orientation of the reference entity; and
predict field of view captured in each of the video frames based on the predicted size and the position of the reference entity.

8. The system according to claim 7, wherein for a predetermined point of view when at least a second portion of the video frames comprises of the reference entity, the one or more processors are configured to anonymize portions of the video frames other than the second portion.

9. The system according to claim 7, wherein for a predetermined point of view when at least a third portion of the video frames comprises of the reference entity, the one or more processors are configured to:
zoom into at least the third portion of video frames; and
anonymize portions of the video frames other than the third portion.

10. The system according to claim 7, wherein for a predetermined field of view when at least a fourth portion of the video frames comprises of the reference entity, the one or more processors are configured to anonymize portions of the video frames other than the fourth portion.

11. The system according to claim 7, wherein for a predetermined field of view when at least a fifth portion of the video frames comprises of the reference entity, the one or more processors are configured to:
zoom into at least the fifth portion of video frames; and
anonymize portions of the video frames other than the fifth portion.

12. The system according to claim 7, wherein for a predetermined field of view and a predetermined point of view the one or more processors are configured to anonymize entire video frames.

13. The system according to claim 7, wherein for a predetermined field of view and a predetermined point of view when a sixth portion of the video frames comprises of the reference entity, the one or more processors are configured to:
anonymize portion of the video frames other than a predetermined area;
the predetermined area is calculated taking the reference entity as a reference point; and
the predetermined area is calculated in both X-axis and Y-axis of the video frames.

14. The system according to claim 5, wherein the one or more processors are configured to analyse the video frames with corrected tag values, wherein the one or more processors are configured to:
compare tag value of each video frame with tag value of immediately preceding video frame;
label a video frame with a first label, when tag values of two adjacent video frames are different, wherein the first label determines change in tag value;
label a video frame with a second label, when tag values of two subsequent adjacent video frames are different, wherein the second label determines change in tag value; and
replace tag values of video frames between the video frame with the first label and the video frame with the second label, wherein the tag values are replaced with tag value of the frame immediately preceding the frame with the first label.

15. The system according to claim 14, wherein the one or more processors are configured to anonymize video frames based on the updated tag values of the video frames.

16. The system according to claim 1, wherein the one or more processors are configured to detect plurality of reference entities when the video frames comprise of multiple reference entities.

17. The system according to claim 16, wherein the one or more processors are configured to:
create a virtual area around the plurality of reference entities, wherein the virtual area covers the plurality of reference entities; and
anonymize portions of the video frames other than the virtual area, wherein the virtual area is determined based on position and orientation of the reference entities.

18. A method for anonymizing videos by a server comprising one or more processors, the method comprising:
receiving a video captured by an imaging device, wherein the video comprises of a plurality of video frames;
analysing each of the video frames captured by the imaging device and detecting reference entity in each of the video frames; and
anonymizing at least a portion of each of the video frames in which the reference entity is absent.

19. The method according to claim 18 comprising:
tagging a video frame with a first value if one or more reference entity is detected in the video frame;
tagging a video frame with a second value if reference entity is absent in the video frame;
identifying incorrectly tagged video frames among the plurality of video frames by analysing tags of immediately preceding and following video frames; and
replacing the value of the incorrectly tagged video frames with a value of one of the preceding or following video frames, wherein:
the incorrectly tagged video frame is determined by inspecting the tagged values of a set of consecutive preceding and following video frames, wherein a video frame is confirmed to be incorrectly tagged when tagged value of the video frame is different from the tagged values of the set of consecutive preceding and following video frames; and
the tagged value of the determined incorrectly tagged video frame is replaced with the value of at least one of the video frames among the inspected set of consecutive preceding and following video frame.

20. The method according to claim 18 comprising:
predicting orientation of the reference entity in each of the video frames;
predicting position of the reference entity in each of the video frames; and
predicting size of the reference entity in each of the video frames.

21. The method according to claim 18 comprising:
zooming into at least a portion of video frames; and
anonymizing portions of the video frames other than portion comprising reference entity, for a predetermined point of view and a predetermined field of view.

22. The system according to claim 19 comprising:
analysing the video frames with corrected tag values;
comparing tag value of each video frame with tag value of immediately preceding video frame;
labelling a video frame with a first label, when tag values of two adjacent video frames are different, wherein the first label determines change in tag value;
labelling a video frame with a second label, when tag values of two subsequent adjacent video frames are different, wherein the second label determines change in tag value; and
replacing tag values of video frames between the video frame with the first label and the video frame with the second label, wherein the tag values are replaced with tag value of the frame immediately preceding the frame with the first label.
